(19) United States
(12) Patent Application Publication
(10) Pub. No.: US 2008/0188854 A1
(43) Pub. Date: Aug. 7, 2008

(54) Surgical Anchor Delivery System

(75) Inventor: Michael William Moser, Gainesville, FL (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: University of Florida Research Foundation, Inc.

(21) Appl. No.: 11/969,595

(22) Filed: Jan. 4, 2008

Related U.S. Application Data
(60) Provisional application No. 60/883,633, filed on Jan. 5, 2007.

(51) Int. Cl. A61B 17/16; A61B 17/17

(52) U.S. Cl. 606/80; 606/96

(57) ABSTRACT

Embodiments of the present invention relate to systems and methods for assisting a user during delivery of a labral anchor. For example, one embodiment provides a labral anchor delivery system that includes a drill sleeve having a proximal end and a distal end that is cannulated therebetween. At least a portion of the drill sleeve proximate to the distal end is curved such that the distal end is configured to be positioned substantially perpendicular to a surface of a glenoid. The labral anchor delivery system includes a drill bit having a proximal end and a distal end and configured to be positioned within the cannulated drill sleeve and into engagement with the surface of the glenoid. In addition, at least a portion of the drill bit between the proximal and distal ends of the drill bit is flexible for conforming to the curved portion of the drill sleeve.

### CROSS REFERENCE TO RELATED APPLICATIONS

The present application claims priority from U.S. Provisional Application No. 60/883,633 filed Jan. 5, 2007, the contents of which are incorporated herein by reference.

### BACKGROUND

Various shoulder injuries may result from dislocations, falling, throwing, or lifting. A common shoulder injury includes the separation of the glenoid labrum from the glenoid. For example, a Bankart lesion results from a labral tear that occurs in the anterioinferior region of the glenoid socket when the shoulder dislocates. A superior labral anterior posterior (SLAP) lesion typically occurs from throwing injuries, where the tear occurs at the superior region of the glenoid socket where the biceps tendon attaches to the shoulder. These injuries result in pain and instability of the shoulder joint.

Arthroscopic stabilization for surgical treatment of shoulder instability has grown in popularity over the past decade. In particular, labral anchors have been employed to repair torn labram tissue. For example, a labral anchor may be inserted into the glenoid, and a suture material that is attached to the labral anchor is used to reattach the torn labral tissue to the glenoid.

Despite advancements in arthroscopic techniques, the failure rate continues to be problematic. Recent articles in the orthopedic literature have discussed the optimal placement of anchor placement on the glenoid that will maximize results. Standard portals used during arthroscopy make it difficult to obtain optimal anchor placement. Other studies have likewise shown that variations from the standard anterior portals place the neurovascular structures at risk.

Therefore, there is a need for systems and methods for assisting a user with the delivery of a labral anchor to reduce the incidence of negative operative and post-operative effects.

### BRIEF SUMMARY

Embodiments of the present invention relate to systems and methods for assisting a user during the delivery of a labral anchor for repairing a labral tear. For example, one embodiment provides a labral anchor delivery system that includes a drill sleeve having a proximal end and a distal end that is cannulated therebetween. In particular embodiments, at least a portion of the drill sleeve proximate to the distal end is curved such that the distal end is configured to be positioned substantially perpendicular to a surface of a glenoid. The labral anchor delivery system includes a drill bit and/or an insertion sleeve. The drill bit has a proximal end and a distal end and is configured to be positioned within the cannulated drill sleeve and into engagement with the surface of the glenoid. In addition, at least a portion of the drill bit between the proximal and distal ends of the drill bit is flexible and is adapted to conform to the curved portion of the drill sleeve. The insertion guide has proximal and distal ends configured to carry a labral anchor, wherein the insertion guide is configured to be positioned within the cannulated drill sleeve and proximate to the surface of the glenoid. Similarly, at least a portion between the proximal and distal ends of the insertion guide is flexible for conforming to the curved portion of the drill sleeve.

According to various aspects of the labral anchor delivery system, the drill sleeve includes a handle proximate to its proximal end. The curved portion of the drill sleeve may be curved with respect to the longitudinal axis of the drill sleeve's promixal end. For instance, the longitudinal axis of the drill sleeve's distal end may form an angle of about 30 degrees with the longitudinal axis of the drill sleeve's proximal end. Moreover, in particular embodiments, the drill bit includes a shank and a plurality of cutting edges and at least a portion of the shank may comprise the flexible portion. The flexible portion of the drill bit may be adjacent to the cutting edges. Furthermore, a labral anchor may be secured to the distal end of the insertion guide, and the flexible portion of the insertion guide may be adjacent to the labral anchor.

### BRIEF DESCRIPTION OF THE DRAWINGS

Having thus described the invention in general terms, reference will now be made to the accompanying drawings, which are not necessarily drawn to scale, and wherein:

FIG. 1 shows an elevational and cross-sectional view of a drill bit positioned within a drill sleeve according to one embodiment of the invention.

FIG. 2 depicts an elevational and cross-sectional view of an insertion guide positioned within a drill sleeve according to an embodiment of the invention.

FIG. 3 depicts an elevational view of a drill sleeve according to a particular embodiment of the invention.

FIG. 3A illustrates an end view of the drill sleeve shown in FIG. 3.

FIG. 4 depicts an elevational view of a drill bit according to one embodiment of the invention.

FIG. 5 depicts an elevational view of an insertion guide according to a particular embodiment of the invention.

### DETAILED DESCRIPTION OF VARIOUS EMBODIMENTS OF THE INVENTION

The present invention will now be described more fully hereinafter with reference to the accompanying drawings, in which various embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Like numbers refer to like elements throughout.

### Overview

Various embodiments of the present invention are configured to assist a user during the delivery of a labral anchor for repairing a labral tear. In particular, certain embodiments of the present invention address the problem associated with fixating a labral anchor in the glenoid or glenoid rim. According to one embodiment of the present invention, a drill sleeve, a drill bit, and an insertion guide are employed to insert a labral anchor into the glenoid for repairing a torn labrum and for restoring shoulder stability. The drill sleeve is cannulated such that the drill bit may be positioned within the drill sleeve, as well as slide into and along the interior of the drill sleeve. The drill bit may include a flexible portion that may be positioned within a curved drill sleeve to guide the drill bit generally perpendicular into the glenoid. In addition, the insertion guide is configured to be positioned within the cannulated drill sleeve and includes a flexible portion that may also be positioned within the curved portion of the drill sleeve so that the insertion guide may deliver a labral anchor into the hole drilled by the drill bit.

### More Detailed Description

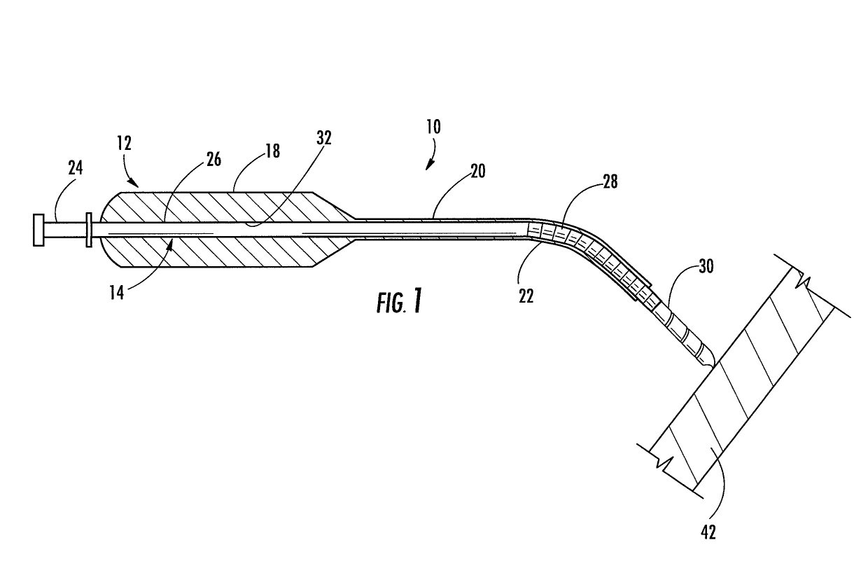

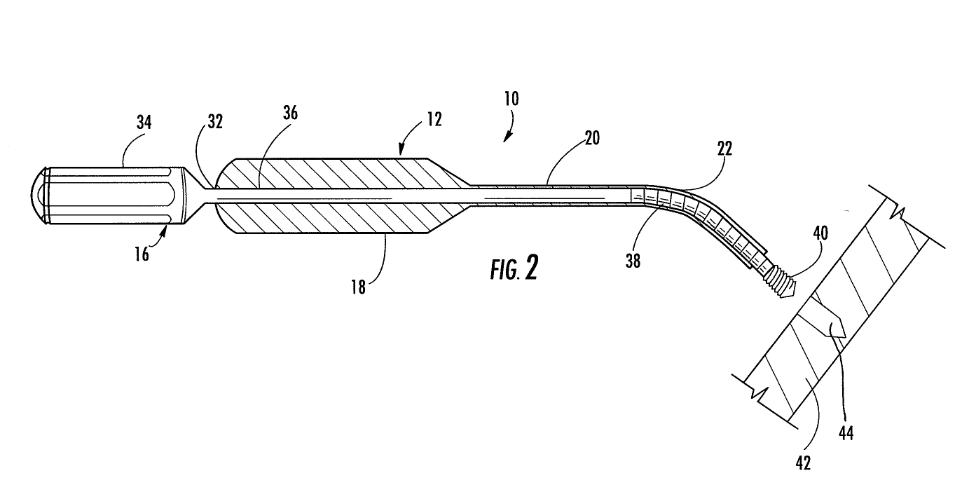

As noted above, various embodiments of the present invention provide techniques for assisting a user during the delivery of a labral anchor into the glenoid. Referring to the figures and, in particular, FIGS. 1 and 2, there is shown a system 10 for assisting a user during delivery of a labral anchor 40, where the system generally includes a drill sleeve 12, a drill bit 14, and an insertion guide 16. The drill sleeve 12 generally includes a curved portion 22, and a flexible portion 28 of the drill bit 14 and a flexible portion 38 of the insertion guide 16 are each respectively adapted to conform to the drill sleeve's curved portion 22 when inserted into the drill sleeve 12. The drill sleeve 12 is employed to position the drill bit substantially perpendicular to the surface of the glenoid 42, while the drill bit 14 drills a hole 44 within the glenoid that is configured to accommodate a labral anchor 40. The insertion guide 16 is used in conjunction with the drill sleeve 12 to deliver the labral anchor 40 into the hole 44 drilled by the drill bit 14. The curved portion 22 and flexible portions 28 and 38 ensure that the labral anchor 40 is inserted substantially perpendicular to the surface of the glenoid 42, which provides for better long-term fixation of the labral anchor.

Generally, embodiments of the system 10 of the invention may be utilized for repairing Bankart lesions and superior labral anterior posterior (SLAP) lesions of the labrum. However, it should be understood that the system 10 may be employed to repair other tissue of the shoulder joint or even tissue of other joints. Thus, the system 10 may be used to guide the drilling of a hole substantially perpendicular to (or at other angles to) the bone of a joint (or other bone or tissue) and deliver an anchor or a screw (or other surgical hardware) into the drilled hole. In addition, one aspect of the system 10 provides for the repair of tears of the rotator cuff. Therefore, although reference is made herein to surgery for repairing labral tears or lesions, such discussion is not meant to be limiting and is used for illustrative purposes only.

### Drill Sleeve

FIGS. 3 and 3A depict a drill sleeve 12 according to one exemplary embodiment of the present invention. FIG. 3 shows that the drill sleeve 12 generally includes a handle 18 proximate to its proximal end, a substantially straight portion 20, and a curved portion 22 proximate to its distal end. As shown in FIG. 3A, at least a portion of the handle 18 has a clover-shaped cross-section (or other suitable cross section) for enhancing gripping of the drill sleeve 12 by a user. FIG. 3A also illustrates that the drill sleeve 12 is cannulated and includes an elongated passageway 32 extending between the proximal and distal ends of the drill sleeve. In addition, FIG. 3A shows that the elongated passageway 32 is generally circular in cross section, although any cross section that is sized and configured to receive the drill bit 14 and insertion guide 16 may be employed. The drill sleeve 12 is typically made of a reusable and rust-proof metallic material that may be sterilized, such as stainless steel. The curved portion 22 could be formed using various techniques such a molding process or bending a straight portion 20 about a form block or mandrel.

In various embodiments, an angle A is defined between the longitudinal axis of the proximal portion of the drill sleeve 12 and the longitudinal axis of the distal portion of the drill sleeve, as shown in FIG. 3. According to one aspect of the invention, the angle A is approximately 30 degrees. This configuration allows a user (e.g., a surgeon) to avoid neurovascular or other structures and position the distal end of the curved portion substantially perpendicular to the glenoid.

It should be understood that the drill sleeve 12 shown in FIGS. 3 and 3A is not meant to be limiting, as the drill sleeve may be various sizes and configurations according to additional aspects of the invention. For example, the curved portion 22 may extend at various angles but is typically within the range of 20-40 degrees. Moreover, the handle 18 may be various sizes and configurations for accommodating different hand sizes and allowing a user to grip and manipulate the drill sleeve 12 to properly position the curved portion 22 proximate to the glenoid. For example, the handle 18 may be configured as a pistol-type grip. Similarly, the diameter of the elongated passageway 32 through the drill sleeve 12 may be various sizes to accommodate (e.g., substantially conform to) various drill bits 14 and insertion guides 16 and to facilitate the placement of labral anchors 40 of different sizes into the glenoid. Furthermore, the term “drill sleeve” is not mean to be limiting, as it should be understood that the drill sleeve may be any sleeve configured to receive various surgical instruments, such as a drill bit 14 and insertion guide 16, as discussed in greater detail below.

### Drill Bit

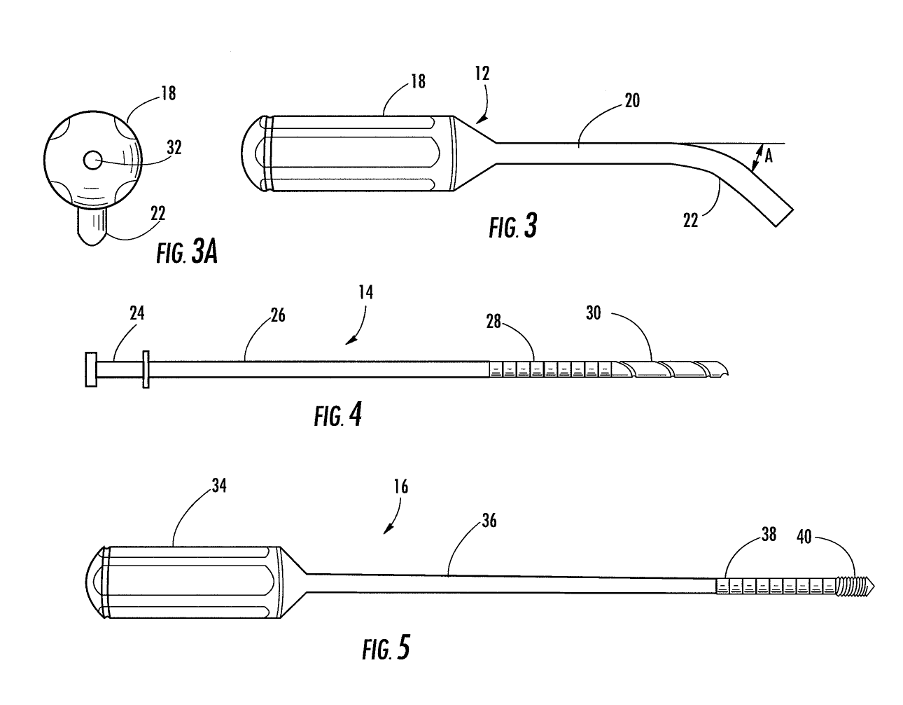

FIG. 4 depicts a drill bit 14 according to an embodiment of the invention. The drill bit 14 is used to drill a hole into the glenoid or glenoid rim. This hole is preferably sized and configured to receive a particular type of labral anchor 40 and to maintain the labral anchor 40 in place due to the frictional engagement between the labral anchor 40 and the portion of the bone defining the hole. The drill bit 14 includes a shank 26 extending from its distal end, a flexible portion 28 adjacent to the shank 26, and a cutting portion 30 adjacent to the flexible portion and proximate to its proximal end of the drill bit. According to one aspect, the shank 26 extends from the distal end and slightly past the midpoint of the drill bit 14, while the flexible portion 28 and the cutting portion 30 comprise the remaining portion of the drill bit. In various embodiments, the flexible portion 28 is located adjacent to the cutting portion 30 such that when the drill bit 14 is axially positioned in operating engagement with the drill sleeve 12, the drill bit's cutting portion 30 extend out of the distal end of the drill sleeve's curved portion 22 while the flexible portion 28 aligns with the curved portion as shown in FIG. 1. Thus, the drill bit 14 is configured to slide proximally and distally within the elongated passageway 32 of the drill sleeve 12, and to contact the glenoid substantially perpendicular to the glenoid. In particular embodiments, at least a portion of the drill bit 14 is configured to substantially conform to the portion of the drill sleeve 12 that defines at least a portion of the elongated passageway 32.

The drill bit 14 is preferably adapted to be attached to a conventional drill (or other suitable rotating device) via a drill chuck (not shown) and driven by the drill (not shown). For instance, the drill chuck could be a three-jaw chuck, a Jacob's chuck, or similar chuck capable of engaging and driving the drill bit 14 during drilling. As shown in FIG. 4, the shank 26 may include an engagement portion 24 proximate to its proximal end for facilitating engagement with the drill chuck. The engagement portion 24 may also include a stop that limits the axial distance that the drill bit 12 may be moved distally within the drill sleeve 12. In various embodiments, the drill bit's cutting portion 30 includes at least one cutting edge this may be helical and converge at a point at the distal end of the drill bit 14. Moreover, the drill may be any suitable device that a user may use to axially and rotatably drive the drill bit 14 in order to penetrate the glenoid substantially perpendicular thereto and remove a desired amount of bone. The drill bit 14 is typically a reusable and rust-proof material that may be sterilized, such as stainless steel. Thus, the flexible portion 28 may be a malleable metallic material or polymeric material (e.g., stainless steel or Kevlar®) that may repetitively bend and conform to the curved portion 22 of the drill sleeve 12.

It is understood that the illustrated drill bit 14 is not meant to be limiting, as the drill bit may be various sizes and configurations in additional aspects of the invention. For example, the diameter and length of the drill bit 14 may be varied in order to drill a hole in the glenoid for different labral anchor 40 sizes depending on user preference or the manufacturer of the labral anchor. For instance, the cutting portion 30 may be about 2 to 3.5 mm in diameter. According to another aspect of the invention, the cutting portion 30 may be adapted to drill within the glenoid to about 5-15 mm in depth, and the drill bit may be about 12 inches in length.

Furthermore, the portion of the drill bit 14 comprising the shank 26 and flexible portion 28 may be the same or similar diameter. For example, the flexible portion 28 may be smaller in diameter than the shank 26 and/or flexible portion 28 to increase the flexibility of the flexible portion 28. Similarly, the flexible portion 28 may comprise a different and more flexible material than the shank 26 and/or cutting portion 30. As such, the drill bit 14 may be an integral structure or an assembly of components such that the flexible portion 28 and cutting portion 30 could be integrally defined in the shank 26 or separately attached to one another. Moreover, all or a portion of the shank 26 may define the flexible portion 28. Thus, the flexible portion 28 may be various lengths but is preferably of sufficient length to ensure that the drill bit 14 may at least substantially conform to the curved portion 22 and that the distal end of the drill bit may contact the glenoid substantially perpendicular thereto. In addition, the drill bit 14 is preferably at least as long as the drill sleeve 12 to ensure that the cutting portion 30 may extend out of the distal end of the drill sleeve. According to one exemplary aspect, the flexible portion 28 and cutting portion 30 are approximately the same length, although any suitable length of flexible portion 28 and cutting portion 30 may be employed.

Insertion Guide

In various embodiments, the system includes an insertion guide 16 that is adapted for facilitating the insertion of surgical hardware (e.g., a surgical anchor or screw) into a hole drilled by the drill bit 14. FIG. 5 illustrates an insertion guide 16 according to one embodiment of the present invention. The insertion guide 16 includes a handle 34 extending from its proximal end, a shaft 36 adjacent the distal end of the handle, a flexible portion 38 adjacent the distal end of the shaft, and a labral anchor 40 adjacent the distal end of the flexible portion (e.g., adjacent the insertion guide's distal end). The insertion guide 16 is configured to be positioned within the elongated passageway 32 of the drill sleeve 12 as shown in FIG. 2, as well as to slide proximally and distally therein. The flexible portion 38 of the insertion guide 16 is configured to align with the drill sleeve's curved portion 22 such that the distal end of the insertion guide 16 may be positioned through the distal end of the drill sleeve 12. Thus, the insertion sleeve 16 is used to deliver the labral anchor 40 into the drilled hole at an angle substantially perpendicular to the surface of the glenoid.

The labral anchor 40 may be any suitable labral anchor known to those of ordinary skill in the art, such as those suitable for repairing labral tears. In various embodiments, the labral anchor 40 may be screwed or hammered into position within the drilled hole—depending on the configuration of the labral anchor. In addition, the labral anchor 40 may be releasably secured to the distal end of the insertion guide 16 using techniques known to those of ordinary skill in the art. For example, the insertion guide 16 may be cannulated, while a suture material attached to a proximal end of the labral anchor may extend through the cannulated insertion guide and out of the proximal end of the insertion guide. The suture material is then tensioned and secured to hold the labral anchor 40 adjacent to the distal end of the insertion guide 16. The manufacturer of the insertion guide 16 may pre-assemble the labral anchor 40 to the insertion guide.

Similar to the drill bit 14, the insertion guide 16 is typically a reusable and rust-proof material that may be sterilized, such as stainless steel. Thus, the flexible portion 38 may be a malleable metallic material or polymeric material (e.g., stainless steel or Kevlar®) that may repetitively bend and conform to the curved portion 22 of the drill sleeve 12. However, the insertion guide 16 may be disposable. When hammering is used to insert the labral anchor 40 in the drilled hole, the insertion guide 16 is preferably a metallic or durable material that may withstand the hammering force and transfer the force to the labral anchor. Moreover, as indicated above with respect to the drill sleeve 12, the handle 34 may be various configurations such as a clover-like configuration or pistol grip for enhancing gripping of the insertion guide 16 by a user.

It should also be understood that the illustrated insertion guide 16 is not meant to be limiting, as the insertion guide may be various sizes and configurations in additional aspects of the invention. The diameter and length of the insertion guide 16 may vary in particular embodiments in order to deliver labral anchors 40 of different sizes depending on user preference or the manufacturer of the labral anchor. For example, the insertion guide 16 may be approximately 12 inches in length but is usually at least as long as the drill sleeve 12 to deliver the labral anchor 40 into a hole drilled in the glenoid. According to one exemplary embodiment, the handle 34, shaft 36, and flexible portion 38 are each approximately a third of the total length of the insertion guide 16, although various lengths may be employed. Moreover, all or a portion of the shaft 36 may be flexible to define the flexible portion 38. Thus, the flexible portion 38 may be various lengths but is preferably of sufficient length to ensure that the insertion guide 16 conforms to the curved portion 22 of the drill sleeve 12. Furthermore, the diameter or material of the flexible portion 38 may differ from the shaft 36. For example, the diameter of the flexible portion 38 may be less than the remaining portion of the shaft 36. In addition, the insertion sleeve 16 may be integrally formed or assembled together such that the flexible portion 38 may be defined in the shaft 36, or the flexible portion may be separately attached to the shaft.

Exemplary Method

As indicated above, embodiments of the system 10 of the invention may be utilized, for example, for repairing Bankart lesions and superior labral anterior posterior (SLAP) lesions. In various embodiments, the system 10 is configured to be used with existing arthroscopic surgical techniques to deliver a labral anchor 40. In this regard, a user may make an anterior incision into which a user inserts the drill sleeve 12. The user then manipulates the drill sleeve's curved portion 22 within the shoulder joint to avoid neurovascular or other structures. A portion of the drill sleeve's substantially straight portion 20 typically extends within the shoulder joint, while the handle 18 remains exterior of the incision for gripping by the user. Next, the user orients the distal end of the curved portion 22 such that the distal end of the curved portion contacts the surface of the glenoid 42 or glenoid rim substantially perpendicular thereto. The curved portion 22 typically contacts the side of the glenoid 42 or glenoid rim proximate to a labral tear. The user may use an endoscope or other imaging technique to aid with the positioning of the drill sleeve 12.

When the user is satisfied with the placement of the drill sleeve 12, the user uses his opposite hand to insert the drill bit 14 within the drill sleeve. As shown in FIG. 1, the user positions the drill bit 14 within the drill sleeve 12 such that the cutting portion 30 extends distally out of the distal end of the drill sleeve's curved portion 22 and contact the glenoid substantially perpendicular to the glenoid. The user may then drill a hole 44 within the glenoid to a desired depth depending on the labral anchor 40 to be inserted. The engagement portion 24 acts as a stop to ensure that the user will drill within the glenoid 42 to a predetermined position in which the user is unable to drill any further. The user could also use an endoscope or other imaging device to determine the depth of drilling.

After the hole 44 has been drilled, the drill bit 14 is removed from the drill sleeve 12. While maintaining the position of the drill sleeve 12 substantially perpendicular to the glenoid 42 and proximate to the drilled hole 44 (see FIG. 2), the user inserts the insertion guide 16 through the drill sleeve 12 as shown in FIG. 2. The user slides the insertion guide 16 through the drill sleeve 12 distally until the labral anchor 40 is positioned proximate to the drilled hole 44. Depending on the type of labral anchor utilized, the user may hammer the proximal end of the insertion guide's handle 34 to deliver the labral anchor into the drilled hole 44, or the user may screw the labral anchor into the drilled hole by rotating the insertion guide's handle 34. The user may use an endoscope or other imaging device to ensure that the labral anchor 40 is completely inserted within the drilled hole 44. The user may then remove the insertion guide 16 and drill sleeve 12 from the subject's shoulder. The user may then use a suture material attached to the proximal end of the labral anchor 40 to lasso and tie down the labral tear using arthroscopic techniques known to those of ordinary skill in the art.

It should be understood that the particular surgical techniques employed to deliver a labral anchor 40 will vary depending on the user's preference and manufacturer of the labral anchor. For example, the subject may be positioned in a beach chair position, while the user may make a 3 or 5 o'clock anterior incision. Thus, the above-described procedure is not meant to be limiting, as the system 10 may be used in a variety of ways to deliver a labral anchor 40 substantially perpendicular to the glenoid.

### CONCLUSION

Many modifications and other embodiments of the invention will come to mind to one skilled in the art to which this invention pertains having the benefit of the teachings presented in the foregoing descriptions and the associated drawings. For example, as will be understood by one skilled in the relevant field in light of this disclosure, the invention may take form in a variety of different mechanical and operational configurations. Therefore, it is to be understood that the invention is not to be limited to the specific embodiments disclosed and that modifications and other embodiments are intended to be included within the scope of the appended exemplary concepts. Although specific terms are employed herein, they are used in a generic and descriptive sense only and not for the purposes of limitation.

What I claim is:

1. A surgical anchor delivery system for delivering a surgical anchor, the system comprising:
a drill sleeve having a proximal end and a distal end, wherein the drill sleeve defines a passage that extends from the proximal end to the distal end, and wherein at least a portion of the drill sleeve proximate to the distal end is curved to allow the distal end to be positioned substantially perpendicular to a surface of a bone into which a portion of the surgical anchor is to be inserted; and
a drill bit having a proximal end and a distal end, wherein the drill bit is configured to be positioned within the passage and into engagement with the surface of the bone, and wherein at least a portion of the drill bit between the drill bit's proximal and distal ends is sufficiently flexible to conform to the curved portion of the drill sleeve.

2. The system of claim 1, wherein the bone is a glenoid.

3. The system of claim 1, wherein the drill sleeve comprises a handle proximate to the drill sleeve's proximal end.

4. The system of claim 1, wherein the proximal end of the drill sleeve defines a longitudinal axis, and wherein the curved portion of the drill sleeve is curved with respect to the longitudinal axis.

5. The system of claim 4, wherein the longitudinal axis of the drill sleeve's proximal end and the longitudinal axis of the drill sleeve's distal end define an angle of about 30 degrees due to the curvature of the drill sleeve's curved portion.

6. The system of claim 1, wherein:
the drill bit comprises a shank and a cutting portion;
the cutting portion defines at least one cutting edge; and
at least a portion of the shank comprises the flexible portion.

7. The system of claim 6, wherein the at least one cutting edge is in a substantially helical configuration.

8. The system of claim 7, wherein the flexible portion of the drill bit is adjacent to the at least one cutting edge.

9. The system of claim 1, further comprising an insertion guide having a proximal end and a distal end, wherein the insertion guide is configured to be positioned within the drill sleeve and proximate to the surface of the bone.

10. The system of claim 9, wherein at least a portion between the proximal and distal ends of the insertion guide is flexible for conforming to the curved portion of the drill sleeve.

11. The system of claim 9, further comprising a labral anchor secured to the distal end of the insertion guide.

12. The system of claim 11, wherein the flexible portion of the insertion guide is adjacent to the labral anchor.

13. A surgical anchor delivery system for delivering a surgical anchor, the system comprising:
a sleeve having a proximal end and a distal end, wherein the sleeve defines a passage that extends from the proximal end to the distal end, and wherein at least a portion of the sleeve proximate to the distal end is curved to allow the distal end to be positioned substantially perpendicular to a surface of a bone into which a portion of the surgical anchor is to be inserted; and
an insertion guide having a proximal end and a distal end, wherein the insertion guide is adapted to both support a surgical anchor and to be positioned within the sleeve proximate to the surface of the bone, wherein at least a portion of the insertion guide between the insertion guide's proximal and distal ends is sufficiently flexible to conform to the sleeve's curved portion.

14. The system of claim 13, wherein the bone is a glenoid.

15. The system of claim 13, wherein the proximal end of the sleeve defines a longitudinal axis, and wherein the curved portion of the sleeve is curved with respect to the longitudinal axis.

16. The system of claim 15, wherein the longitudinal axis of the sleeve's proximal end and the longitudinal axis of the sleeve's distal end define an angle of about 30 degrees due to the curvature of the sleeve's curved portion.

17. The system of claim 13, further comprising a drill bit having a proximal end and a distal end, wherein the drill bit is configured to be positioned within the sleeve's passage and into engagement with the surface of the bone.

18. The system of claim 16, wherein at least a portion of the drill bit between the proximal and distal ends of the drill bit is sufficiently flexible to conform to the curved portion of the sleeve.

19. The system of claim 13, wherein the flexible portion of the insertion guide is adjacent to a surgical anchor that is supported by the insertion guide.

20. A method for installing a surgical anchor comprising:
positioning a drill sleeve so that a distal end of the drill sleeve is substantially perpendicular to a surface of a bone, wherein at least a portion of the drill sleeve proximate to the distal end is curved;
sliding a drill bit into a passage defined by the drill sleeve so that, as a flexible portion of the drill bit slides into the drill sleeve's curved portion, the flexible portion bends into a curved orientation to at least generally conform to the shape of the drill sleeve's curved portion;
while the drill bit's flexible portion is in the curved orientation, using the drill bit to drill a hole in the bone; and
after drilling the hole, inserting the surgical anchor into the hole.

21. The method of claim 20, wherein the step of drilling comprises drilling the hole substantially perpendicular to the surface of the bone.

22. The method of claim 20, wherein the bone is a glenoid.

23. The method of claim 20, wherein the step of inserting the surgical anchor into the hole comprises sliding an insertion guide to which the surgical anchor is attached into the passage defined by the drill sleeve until the surgical anchor is in place within the hole.

24. The method of claim 23, wherein the step of sliding the insertion guide comprises sliding the insertion guide into the passage defined by the drill sleeve so that, as a flexible portion of the insertion guide slides into the drill sleeve's curved portion, the flexible portion of the insertion guide bends into a curved orientation to at least generally conform to the shape of the drill sleeve's curved portion.

25. The method of claim 23, further comprising removing the drill bit from the drill sleeve prior to inserting the insertion sleeve into the drill sleeve.
